(19) United States
(12) Patent Application Publication
(10) Pub. No.: US 2022/0055971 A1
(43) Pub. Date: Feb. 24, 2022

(54) METHOD OF METHYL CYCLOPENTENE PRODUCTION FROM CYCLOHEXENE OVER ZEOLITE-BASED CATALYST STRUCTURE

(71) Applicant: Kara Technologies Inc., Calgary AB (CA)

(72) Inventors: Hua Song, Calgary (CA); Hao Xu, Calgary (CA); Zhaofei Li, Calgary (CA)

(21) Appl. No.: 17/404,052

(22) Filed: Aug. 17, 2021

Related U.S. Application Data
(60) Provisional application No. 63/066,945, filed on Aug. 18, 2020.

(51) Int. Cl. C07C 5/29; B01J 29/40; B01J 29/48; B01J 29/44

(52) U.S. Cl. CPC C07C 5/29; B01J 29/40; B01J 29/405; B01J 29/48; C07C 2529/48; C07C 2529/40; C07C 2523/50; C07C 2523/08; B01J 29/44

(57) ABSTRACT

Selective conversion from cyclohexene to methylcyclopentene can occur via skeletal isomerization reaction under mild temperature and near atmospheric pressure with the existence of a catalyst structure as described herein. The catalyst structure includes a porous zeolite as the support and one or more loaded metals to further modify its acidity and pore structures. Industrially available cyclohexene feedstock can be effectively converted to a high value-added product methylcyclopentene with over 90 wt % conversion and 95 wt % selectivity, which is highly profitable for potential application in the fine chemical industry.

### CROSS-REFERENCE TO RELATED APPLICATIONS

The present application claims priority from U.S. Provisional Patent Application Ser. No. 63/066,945, filed Aug. 18, 2020 and entitled “Method of Methyl Cyclopentene Production From Cyclohexene Over Zeolite-Based Catalyst Structure”, the disclosure of which is incorporated herein by reference in its entirety.

### FIELD

The present invention relates to the field of fine chemical processing.

### BACKGROUND

Currently, alternative and sustainable energy sources have become very attractive due to the limited reserve of crude oil resources. Catalytic pyrolysis is one of the most used techniques for the enhancement of oil qualities, especially for the generation of bio-derived fuels from various types of biomass. The quality of oil products can be greatly improved with the cracking of heavy crudes to achieve light oil as well as the removal of heterogeneous atoms. Therefore, the corresponding study is of great importance for sustainable energy development, especially in the current situation of foreseeable fossil fuel depletion.

Cyclohexene is one of the most common components in the light oil pyrolysis process, which can be directly produced by pyrolysis of rice straw or other crude oils. However, its value is underestimated due to limited applications. Based upon the chemical activity and mutability due to the properties of both olefin and cyclohydrocarbons, it is anticipated that a series of following reactions may be conducted during the catalytic cyclohexene conversion process including ring-opening, cracking, isomerization, alkylation, aromatization and hydrogenation. It is known that the products via catalytic cyclohexene conversion can be various types of hydrocarbons, including LPG such as ethylene, propane, propylene and 1,3-butadiene, liquid products such as cyclohexane, cyclohexadiene and benzene, as well as heavier polyaromatic hydrocarbons, and even coke. However, high selectivity toward a specific product is often difficult to achieve due to the complexity of the reaction network. Therefore, the establishment of a strategy to get better control of the catalytic cyclohexene pyrolysis process, namely improving the selectivity of specific desired products with high value such as methylcyclopentene, is of great significance and for potential industrial applications.

Methylcyclopentene (mcp), as an isomer of cyclohexene, can be produced from cyclohexene through a skeletal isomerization reaction. In contrast to cyclohexene, it is specifically valuable for the synthesis of a series of chemical derivatives with great demand from petrochemical refineries. For example, mcp can form polyolefins together with ethylene with unique properties such as high mechanical strength, corrosion resistance and electrical conductivity. It is also used for the synthesis of various insecticides, resin intermediates, and related products. Therefore, mcp has a much higher market value compared to cyclohexene.

The conventional industrial approach for producing methylcyclopentene mainly relies on the dehydrogenation of methylcyclopentane, but the activity of catalysts is always too low for efficient production since methyl cyclopentane is quite stable. In addition, benzene is also widely observed as an unfavorable by-product, and the resulting separation process causes increased capital and operational cost and complexity. The market price of methylcyclopentene, especially 1-methylcyclopentene, is much higher than other isomers. Therefore, it is highly profitable if methylcyclopentene with high selectivity can be produced through a facile and low-cost process from readily available sources, and the selective conversion of cyclohexene toward methylcyclopentene through skeletal isomerization reaction under mild conditions is thus economically appealing.

The traditional catalysts for cyclohexene skeletal isomerization are mainly based on metal oxides including SiO₂, Al₂O₃ and ZrO₂, or their combinations. It is widely reported and accepted that the activity of those metal oxide catalysts is correlated to increasing surface acidity. However, the highly active catalysts with high surface acidity inevitably lead to a variety of side reactions including hydrogen transfer, cracking and coking, lowering the selectivity toward methylcyclopentene. On the other hand, the modified catalyst with low surface acidity always demonstrates insufficient activity with low conversion. Therefore, the industrial applications of these catalysts are hindered due to poor profitability. New catalyst structures with both high cyclohexene conversion and high selectivity to desired products are crucial for real industrial applications.

### BRIEF SUMMARY

In accordance with example embodiments described herein, a method for producing methylcyclopentene from cyclohexene via skeletal isomerization comprises reacting cyclohexene within a reactor in the presence of a gas atmosphere and a catalyst structure, where the catalyst structure comprises a porous support structure and one or more metals loaded in the porous support structure, the porous support structure comprises an aluminosilicate material, and the one or more metals loaded in the porous support structure is selected from the group consisting of Na, K, Co, Mo, Ag, Ga and Ce.

In certain embodiments, each metal loaded in the porous support structure is present in an amount from about 0.1 wt % to about 20 wt %.

The implementation of the embodiments can further result in advantageous catalytic performances including but not limited to: over 97 wt % olefin selectivity, over 95 wt % methylcyclopentene selectivity, over 90 wt % cyclohexene conversion, below 0.5 wt % gas yield, below 0.5 wt % coke yield, long catalyst lifetime.

The above and still further features and advantages of the present invention will become apparent upon consideration of the following detailed description of specific embodiments thereof.

### DETAILED DESCRIPTION

In the following detailed description, while aspects of the disclosure are disclosed, alternate embodiments of the present disclosure and their equivalents may be devised without parting from the spirit or scope of the present disclosure. It should be noted that any discussion herein regarding “one embodiment”, “an embodiment”, “an exemplary embodiment”, and the like indicate that the embodiment described may include a particular feature, structure, or characteristic, and that such particular feature, structure, or characteristic may not necessarily be included in every embodiment. In addition, references to the foregoing do not necessarily comprise a reference to the same embodiment. Finally, irrespective of whether it is explicitly described, one of ordinary skill in the art would readily appreciate that each of the particular features, structures, or characteristics of the given embodiments may be utilized in connection or combination with those of any other embodiment discussed herein.

Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiment. Various additional operations may be performed and/or described operations may be omitted in additional embodiments.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B and C).

The terms “comprising,” “including,” “having,” and the like, as used with respect to embodiments of the present disclosure, are synonymous.

The present invention relates to a method to produce methylcyclopentene from cyclohexene via a skeletal isomerization reaction. In particular, the present invention is directed toward the formulation of a heterogeneous catalyst and a process of utilizing the catalyst for isomerization reaction at mild conditions to produce high value-added products in an efficient and effective manner.

Currently, a catalyst with both high activity to convert cyclohexene and methylcyclopentene selectivity does not appear commercially available or known in the literature. In accordance with the present invention, a series of zeolite-based catalysts are provided for triggering cyclohexene conversion under industrially favorable mild conditions from about 350° C. to about 450° C. (e.g., about 400° C.) and near atmospheric or low pressure (e.g., about 1 atm to about 35 atm). It is observed that the highly selective skeletal isomerization of cyclohexene to produce methylcyclopentene with high purity can be realized via a zeolite-based catalyst structure as described herein with rational acidity modifications. This method provides an isomerization approach for light oil conversion to achieve more valuable products in the petroleum industry. In particular, the outcomes derived from the disclosure herein present an economically and operationally promising route for the production of methylcyclopentene with high purity from abundant resources.

In accordance with example embodiments, catalyst structures are described herein for use in the skeletal isomerization process to produce methylcyclopentene from cyclohexene under near atmospheric pressure of N₂, Ar, etc. with catalyst structure to achieve high value-added chemicals.

The design of the catalyst so as to trigger the molecular re-engineering and selectively formation of the desired products is very important to the efficacy of the process. Particularly, the catalyst structures described herein facilitate the skeletal isomerization process at mild conditions (e.g., in the range of 350-450° C., preferably 400° C.) and near atmospheric pressures (e.g., in the range of 1-35 atm, such as around 1 atm) and in the presence of a catalyst structure.

Catalyst Structures

In accordance with the present invention, a catalyst structure is provided that comprises one or the combination of more than one metallic active components loaded on highly porous supports such as zeolite for cyclohexene isomerization to achieve desired product methylcyclopentene at near atmospheric pressure.

A suitable highly porous support material (also referred to as a porous catalyst support structure) can be an aluminosilicate such as zeolite material. Some non-limiting examples of a suitable zeolite material as a support for the catalyst structure include HZSM-5 type zeolite, L-type zeolite, HX type zeolite, HY type zeolite, etc. For example, a home-made uniform ZSM-5 can be made as described in some of the examples herein.

Suitable metals that can be loaded on the porous support material by impregnation or doping include one or more from the following group: Na, K, Co, Mo, Ag, Ga and Ce. Each metal dopant or the combination of metal dopants can be provided within the catalyst structure in an amount ranging from 0.1-20 wt % (i.e., based upon the total weight of the catalyst structure). Specific examples of different metal loadings for catalyst structures are provided herein.

The porous support material can be doped with a suitable amount of one or more metals in the following manner. One or more metal salts can be dissolved in deionized water to form an aqueous solution at suitable concentration(s) within the solution. Metal precursor salts that can be used to form the catalyst structure include, without limitation, hydroxides, chlorides and nitrates. The one or more metal precursors in solution are then loaded into the porous support material to achieve a desired amount of metals within the catalyst structure (e.g., from 0.1-20 wt %). Any suitable loading process can be performed to load metals within the porous support material. Some non-limiting examples of metal loading processes include: IWI (incipient wetness impregnation, where an active metal precursor is first dissolved in an aqueous or organic solution, the metal-containing solution is then added to a catalyst support containing the same pore volume as the added solution volume, where capillary action draws the solution into the pores); WI (wet impregnation, where more liquid than the IWI volume is added to the support, and the solvent is then removed by evaporation); IE (ion-exchange, where metal cations are exchanged into the support from solution); and FI (framework incorporation, where metals are added to the support materials during the synthesis step of the support).

Depending upon the particular loading process, the resultant metal-loaded catalyst structure can be dried at a temperature between about 80° C. to about 120° C. for a period of time between about 2 hours to about 24 hours. The dried catalyst structure can then be calcined under air, N₂, He or Ar gas at a temperature ranging from 300-700° C. and a suitable ramped or stepped increased heating rate (e.g., about 5-20° C./min), where such calcination temperatures, times and heating rates can be modified depending upon the type or types of metals doped into the catalyst structure as well as reaction conditions associated with the use of the catalyst structure.

The resultant metal-doped catalyst structure is suitable for use in cyclohexene skeletal isomerization reaction under near atmospheric pressure in processes as described herein. The catalyst structure can be processed into a granular form with a granule size desired for a particular operation. The catalyst structure can also be formed into any other suitable configuration. For example, the catalyst structure in a powder form can be utilized in a batch reactor system, while the catalyst structure in a pelleted form can be utilized in a fixed bed continuous flow reactor system (e.g., a continuous tubular reactor (CTR)).

Systems and Methods for Cyclohexene Conversion Utilizing the Catalyst Structures

The skeletal isomerization conversion of cyclohexene and selectivity toward valuable methylcyclopentene can be fine-tuned using catalyst structures as described herein and under a specific gas environment. Different reactor systems and modified operating conditions (e.g., temperatures and pressures), as well as modifications of the catalyst structures within the reactor systems, can also be implemented to achieve varied product compositions.

In a batch or continuous reactor system, a mass ratio of cyclohexene to catalyst structure can be provided within a range of about 100:1 to about 1:1 to achieve a desired yield of methylcyclopentene (mcp) and/or other olefins. Within a continuous system (e.g., a CTR), a liquid hourly space velocity (LHSV) of the cyclohexene can be provided within the range of 1 h⁻¹ to 100 h⁻¹.

The skeletal isomerization process as described herein can be very promising in the industry as the conversion of cyclohexene to reaction products exceeds 50 wt %, or even 90 wt % or greater. The process also can have a selectivity toward methylcyclopentene that exceeds 50 wt %, or even 95 wt % or greater, where the selectivity remains unchanged after 5 runs, which proves the durability of this catalyst structure. The process can also achieve over 97 wt % olefin selectivity, below 0.5 wt % gas yield, and below 0.5 wt % coke yield, with a long catalyst lifetime.

Some examples of the skeletal isomerization from cyclohexene using the aforementioned catalyst structure and process are now described.

### Example 1

An NH₄-ZSM-5 (SiO₂:Al₂O₃=23:1) catalyst structure in powder form was obtained from Zeolyst USA. The catalyst was calcined in N₂ at 600° C. for 3 hours to get the HZSM-5(23:1) catalyst.

The catalytic cyclohexene conversion was carried out under a CH₄ atmosphere in a batch reactor at 400° C. and 30 bar for 1 hour. First, 0.0200 g HZSM-5(23:1) catalyst and 2.00 g cyclohexene were put in a 100 mL batch reactor. Then, the reactor was sealed by flange with a reactor cap. Next, CH₄ was used to purge the reactor three times through a connected tubing system so that the other components in the air could be fully removed from the reactor. Subsequently, the reactor was set into a heating mantle and heated up to 400° C. after passing a leak test. The heating up stage took about 20 minutes and the reaction lasted another 1 hour after the temperature reached the set point. When the scheduled reaction time was met, the reactor was cooled down by blowing air flow at room temperature. The overall analysis, PONA (paraben, olefin, naphtha, aromatic) selectivity and specific olefin selectivity are listed in Table 1, Table 2 and Table 3 respectively.

### Example 2

An NH₄-ZSM-5 (SiO₂:Al₂O₃=80:1) catalyst structure in powder form was obtained from Zeolyst USA. The catalyst was calcined in He at 600° C. for 3 hours to get the HZSM-5(80:1) catalyst.

The catalytic cyclohexene conversion was carried out under a CH₄ atmosphere in a batch reactor at 450° C. and 30 bar for 1 hour. First, 2.0000 g HZSM-5(80:1) catalyst and 2.00 g cyclohexene were put in a 100 mL batch reactor. Then, the reactor was sealed by flange with a reactor cap. Next, CH₄ was used to purge the reactor three times through a connected tubing system so that the other components in the air could be fully removed from the reactor. Subsequently, the reactor was set into a heating mantle and heated up to 450° C. after passing a leak test. The heating up stage took about 20 minutes and the reaction lasted another 1 hour after the temperature reached the set point. When the scheduled reaction time was met, the reactor was cooled down by blowing air flow at room temperature. The overall analysis, PONA selectivity and specific olefin selectivity are listed in Table 1, Table 2 and Table 3 respectively.

### Example 3

An NH₄-ZSM-5 (SiO₂:Al₂O₃=280:1) catalyst structure in powder form was obtained from Zeolyst USA. The catalyst was calcined in Ar at 600° C. for 3 hours to get the HZSM-5(280:1) catalyst.

The catalytic cyclohexene conversion was carried out under a CH₄ atmosphere in a batch reactor at 400° C. and 30 bar for 1 hour. First, 0.2000 g HZSM-5(280:1) catalyst and 2.00 g cyclohexene were put in a 100 mL batch reactor. Then, the reactor was sealed by flange with a reactor cap. Next, CH₄ was used to purge the reactor three times through a connected tubing system so that the other components in the air could be fully removed from the reactor. Subsequently, the reactor was set into a heating mantle and heated up to 400° C. after passing a leak test. The heating up stage took about 20 minutes and the reaction lasted another 1 hour after the temperature reached the set point. When the scheduled reaction time was met, the reactor was cooled down by blowing air flow at room temperature. The overall analysis, PONA selectivity and specific olefin selectivity are listed in Table 1, Table 2 and Table 3 respectively.

### Example 4

An NH₄-ZSM-5 (SiO₂:Al₂O₃=23:1) catalyst structure in powder form was obtained from Zeolyst USA. The catalyst was calcined in static air at 600° C. for 3 hours to get the HZSM-5(23:1). The following metal salts were dissolved in deionized water to form a metal precursor solution: Ga(NO₃)₃.9H₂O and AgNO₃. The HZSM-5 support was impregnated with the metal precursor solution to achieve a suitable metal weight loading of 1 wt % Ag and 1 wt % Ga. The obtained wet powder was first dried in an oven at 90° C. overnight, followed by calcination at 550° C. in static air for 2 hours to get the 1Ag1Ga/ZSM-5 catalyst.

The catalytic cyclohexene conversion was carried out under a CH₄ atmosphere in a batch reactor at 400° C. and 30 bar for 1 hour. First, 0.2000 g 1 Ag1Ga/ZSM-5 catalyst and 2.00 g cyclohexene were put in a 100 mL batch reactor. Then, the reactor was sealed by flange with a reactor cap. Next, CH₄ was used to purge the reactor three times through a connected tubing system so that the other components in the air could be fully removed from the reactor. Subsequently, the reactor was set into a heating mantle and heated up to 400° C. after passing a leak test. The heating up stage took about 20 minutes and the reaction lasted another 1 hour after the temperature reached the set point. When the scheduled reaction time was met, the reactor was cooled down by blowing air flow at room temperature. The overall analysis, PONA selectivity and specific olefin selectivity are listed in Table 1, Table 2 and Table 3 respectively.

### Example 5

An NH₄-ZSM-5 (SiO₂:Al₂O₃=23:1) catalyst structure in powder form was obtained from Zeolyst USA. The catalyst was calcined in static air at 600° C. for 3 hours to get the HZSM-5(23:1). The following metal salts were dissolved in deionized water to form a metal precursor solution: (NH₄)₆Mo₇O₂₄.4H₂O. The HZSM-5 support was impregnated with the metal precursor solution to achieve a suitable metal weight loading of 20 wt % Mo. The obtained wet powder was first dried in an oven at 90° C. overnight, followed by calcination at 550° C. in static air for 2 hours to get the 20 Mo/ZSM-5 catalyst.

The catalytic cyclohexene conversion was carried out under a CH₄ atmosphere in a batch reactor at 400° C. and 30 bar for 1 hour. First, 0.2000 g 20Mo/ZSM-5 catalyst and 2.00 g cyclohexene were put in a 100 mL batch reactor. Then, the reactor was sealed by flange with a reactor cap. Next, CH₄ was used to purge the reactor three times through a connected tubing system so that the other components in the air could be fully removed from the reactor. Subsequently, the reactor was set into a heating mantle and heated up to 400° C. after passing a leak test. The heating up stage took about 20 minutes and the reaction lasted another 1 hour after the temperature reached the set point. When the scheduled reaction time was met, the reactor was cooled down by blowing air flow at room temperature. The overall analysis, PONA selectivity and specific olefin selectivity are listed in Table 1, Table 2 and Table 3 respectively.

### Example 6

An NH₄-ZSM-5 (SiO₂:Al₂O₃=23:1) catalyst structure in powder form was obtained from Zeolyst USA. The catalyst was calcined in static air at 600° C. for 3 hours to get the HZSM-5(23:1). The following metal salts were dissolved in deionized water to form a metal precursor solution: (NH₄)₆Mo₇O₂₄.4H₂O, Co(NO₃)₂.6H₂O. The HZSM-5 support was impregnated with the metal precursor solution to achieve a suitable metal weight loading of 20 wt % Mo and 7.4 wt % Co. The obtained wet powder was first dried in an oven at 80° C. overnight, followed by calcination at 700° C. in static air for 2 hours to get the 20Mo7.4Mo/ZSM-5 catalyst.

The catalytic cyclohexene conversion was carried out under a CH₄ atmosphere in a batch reactor at 400° C. and 30 bar for 1 hour. First, 0.2000 g 20Mo7.4Co/ZSM-5 catalyst and 2.00 g cyclohexene were put in a 100 mL batch reactor. Then, the reactor was sealed by flange with a reactor cap. Next, CH₄ was used to purge the reactor three times through a connected tubing system so that the other components in the air could be fully removed from the reactor. Subsequently, the reactor was set into a heating mantle and heated up to 400° C. after passing a leak test. The heating up stage took about 20 minutes and the reaction lasted another 1 hour after the temperature reached the set point. When the scheduled reaction time was met, the reactor was cooled down by blowing air flow at room temperature. The overall analysis, PONA selectivity and specific olefin selectivity are listed in Table 1, Table 2 and Table 3 respectively.

### Example 7

An NH₄-ZSM-5 (SiO₂:Al₂O₃=23:1) catalyst structure in powder form was obtained from Zeolyst USA. The catalyst was calcined in static air at 600° C. for 3 hours to get the HZSM-5(23:1). The following metal salts were dissolved in deionized water to form a metal precursor solution: (NH₄)₆Mo₇O₂₄.4H₂O, Ce(NO₃)₃.6H₂O. The HZSM-5 support was impregnated with the metal precursor solution to achieve a suitable metal weight loading of 20 wt % Mo and 10 wt % Ce. The obtained wet powder was first dried in an oven at 120° C. overnight, followed by calcination at 300° C. in static air for 2 hours to get the 20Mo10Ce/ZSM-5 catalyst.

The catalytic cyclohexene conversion was carried out under a CH₄ atmosphere in a batch reactor at 400° C. and 30 bar for 1 hour. First, 0.2000 g 20Mo10Ce/ZSM-5 catalyst and 2.00 g cyclohexene were put in a 100 mL batch reactor. Then, the reactor was sealed by flange with a reactor cap. Next, CH₄ was used to purge the reactor three times through a connected tubing system so that the other components in the air could be fully removed from the reactor. Subsequently, the reactor was set into a heating mantle and heated up to 400° C. after passing a leak test. The heating up stage took about 20 minutes and the reaction lasted another 1 hour after the temperature reached the set point. When the scheduled reaction time was met, the reactor was cooled down by blowing air flow at room temperature. The overall analysis, PONA selectivity and specific olefin selectivity are listed in Table 1, Table 2 and Table 3 respectively.

### Example 8

An NH₄-ZSM-5 (SiO₂:Al₂O₃=23:1) catalyst structure in powder form was obtained from Zeolyst USA. The catalyst was calcined in static air at 600° C. for 3 hours to get the HZSM-5(23:1). The following metal salts were dissolved in deionized water to form a metal precursor solution: (NH₄)₆Mo₇O₂₄.4H₂O, Co(NO₃)₂.6H₂O, Ce(NO₃)₃.6H₂O, Ga(NO₃)₃.9H₂O and AgNO₃. The HZSM-5 support was impregnated with the metal precursor solution to achieve a suitable metal weight loading of 20 wt % Mo, 7.4 wt % Co, 10 wt % Ce, 0.1 wt % Ag and 0.1 wt % Ga. The obtained wet powder was first dried in an oven at 90° C. overnight, followed by calcination at 550° C. in static air for 2 hours to get the 20Mo10Ce7.4Co0.1Ag0.1Ga/ZSM-5 catalyst.

The catalytic cyclohexene conversion was carried out under a CH₄ atmosphere in a batch reactor at 400° C. and 30 bar for 1 hour. First, 0.2000 g 20Mo10Ce7.4Co0.1Ag0.1Ga/ZSM-5 catalyst and 2.00 g cyclohexene were put in a 100 mL batch reactor. Then, the reactor was sealed by flange with a reactor cap. Next, CH₄ was used to purge the reactor three times through a connected tubing system so that the other components in the air could be fully removed from the reactor. Subsequently, the reactor was set into a heating mantle and heated up to 400° C. after passing a leak test. The heating up stage took about 20 minutes and the reaction lasted another 1 hour after the temperature reached the set point. When the scheduled reaction time was met, the reactor was cooled down by blowing air flow at room temperature. The overall analysis, PONA selectivity and specific olefin selectivity are listed in Table 1, Table 2 and Table 3 respectively.

### Example 9

A home-made uniform zeolite catalyst UZSM-5 with SiO₂ to Al₂O₃ molar ratio of 80:1 was prepared in the following procedures. 120 g tetraethyl orthosilicate was firstly added dropwise through a burette into a solution containing 100 mL 0.1 mol L⁻¹ tetrapropylammonium hydroxide and 5.4020 g aluminum nitrate. Then, the solution was further stirred and put in a 500 mL teflon lined autoclave. Next, the autoclave was sealed and heated up to 170° C. and held for 72 hours for hydrothermal synthesis. After the hydrothermal treatment, the product was collected and rinsed three times by deionized water through centrifugation. Finally, the catalyst structure was obtained through calcination at 600° C. in static air for 3 hours to get UZSM-5.

The following metal salts were dissolved in deionized water to form a metal precursor solution: (NH₄)₆Mo₇O₂₄.4H₂O and Co(NO₃)₂.6H₂O. The UZSM-5 support was impregnated with the metal precursor solution to achieve a suitable metal weight loading of 20 wt % Mo and 10 wt % Co. The obtained wet powder was first dried in an oven at 90° C. overnight, followed by calcination at 550° C. in static air for 2 hours to get the 20Mo10Co/UZSM-5 catalyst.

The catalytic cyclohexene conversion was carried out in a batch reactor at 400° C. under a gas atmosphere of 32 bar CH₄ and 3 bar N₂ for 1 hour. First, 0.2000 g 20Mo10Co/UZSM-5 catalyst and 2.00 g cyclohexene were put in a 100 mL batch reactor. Then, the reactor was sealed by flange with a reactor cap. Next, CH₄ was used to purge the reactor three times through a connected tubing system so that the other components in the air could be fully removed from the reactor. Subsequently, the reactor was set into a heating mantle and heated up to 400° C. after passing a leak test. The heating up stage took about 20 minutes and the reaction lasted another 1 hour after the temperature reached the set point. When the scheduled reaction time was met, the reactor was cooled down by blowing air flow at room temperature. The overall analysis, PONA selectivity and specific olefin selectivity are listed in Table 1, Table 2 and Table 3 respectively.

### Example 10

A home-made uniform zeolite catalyst UZSM-5 with SiO₂ to Al₂O₃ molar ratio of 80:1 was prepared in the following procedures. 120 g tetraethyl orthosilicate was firstly added dropwise through a burette into a solution containing 100 mL 0.1 mol L⁻¹ tetrapropylammonium hydroxide and 5.4020 g aluminum nitrate. Then, the solution was further stirred and put in a 500 mL teflon lined autoclave. Next, the autoclave was sealed and heated up to 170° C. and held for 72 hours for hydrothermal synthesis. After the hydrothermal treatment, the product was collected and rinsed three times by deionized water through centrifugation. Finally, the catalyst structure was obtained through calcination at 600° C. in static air for 3 hours to get UZSM-5. NaUZSM-5 catalyst was prepared from UZSM-5 through an ion exchange process. In a typical ion exchange cycle, 1 g UZSM-5 was put in 50 mL 0.01 mol L⁻¹ NaOH aqueous solution and set for 5 minutes at room temperature, followed by a centrifugation process to separate the catalyst and the solution. The ion exchange cycle was repeated three times, and the final product was fully rinsed by deionized water and calcined at 600° C. in static air for 3 hours to get NaUZSM-5.

The following metal salts were dissolved in deionized water to form a metal precursor solution: (NH₄)₆Mo₇O₂₄.4H₂O and Co(NO₃)₂.6H₂O. The NaUZSM-5 support was impregnated with the metal precursor solution to achieve a suitable metal weight loading of 20 wt % Mo and 10 wt % Co. The obtained wet powder was first dried in an oven at 90° C. overnight, followed by calcination at 550° C. in static air for 2 hours to get the 20Mo10Co/NaUZSM-5 catalyst.

The catalytic cyclohexene conversion was carried out in a batch reactor at 400° C. under a gas atmosphere of 32 bar CH₄ and 3 bar N₂ for 1 hour. First, 0.2000 g 20Mo10Co/NaUZSM-5 catalyst and 2.00 g cyclohexene were put in a 100 mL batch reactor. Then, the reactor was sealed by flange with a reactor cap. Next, CH₄ was used to purge the reactor three times through a connected tubing system so that the other components in the air could be fully removed from the reactor. Subsequently, the reactor was set into a heating mantle and heated up to 400° C. after passing a leak test. The heating up stage took about 20 minutes and the reaction lasted another 1 hour after the temperature reached the set point. When the scheduled reaction time was met, the reactor was cooled down by blowing air flow at room temperature. The overall analysis, PONA selectivity and specific olefin selectivity are listed in Table 1, Table 2 and Table 3 respectively.

### Example 11

An NH₄-ZSM-5 (SiO₂:Al₂O₃=80:1) catalyst structure in powder form was obtained from Zeolyst USA. The catalyst was calcined in static air at 600° C. for 3 hours to get the HZSM-5(80:1) catalyst. NaZSM-5 catalyst was prepared from ZSM-5 through an ion exchange process. In a typical ion exchange cycle, 1 g ZSM-5 was put in 50 mL 0.01 mol L⁻¹ NaOH aqueous solution and set for 5 minutes at room temperature, followed by a centrifugation process to separate the catalyst and the solution. The ion exchange cycle was repeated three times, and the final product was fully rinsed by deionized water and calcined at 600° C. in static air for 3 hours to get NaZSM-5.

The following metal salts were dissolved in deionized water to form a metal precursor solution: (NH₄)₆Mo₇O₂₄.4H₂O and Co(NO₃)₂.6H₂O. The NaZSM-5 support was impregnated with the metal precursor solution to achieve a suitable metal weight loading of 20 wt % Mo and 10 wt % Co. The obtained wet powder was first dried in an oven at 90° C. overnight, followed by calcination at 550° C. in static air for 2 hours to get the 20Mo10Co/NaZSM-5 catalyst.

The catalytic cyclohexene conversion was carried out in a batch reactor at 400° C. under a gas atmosphere of 32 bar CH₄ and 3 bar N₂ for 1 hour. First, 0.2000 g 20Mo10Co/NaZSM-5 catalyst and 2.00 g cyclohexene were put in a 100 mL batch reactor. Then, the reactor was sealed by flange with a reactor cap. Next, CH₄ was used to purge the reactor three times through a connected tubing system so that the other components in the air could be fully removed from the reactor. Subsequently, the reactor was set into a heating mantle and heated up to 400° C. after passing a leak test. The heating up stage took about 20 minutes and the reaction lasted another 1 hour after the temperature reached the set point. When the scheduled reaction time was met, the reactor was cooled down by blowing air flow at room temperature. The overall analysis, PONA selectivity and specific olefin selectivity are listed in Table 1, Table 2 and Table 3 respectively.

### Example 12

A home-made uniform zeolite catalyst UZSM-5 with SiO₂ to Al₂O₃ molar ratio of 80:1 was prepared in the following procedures. 120 g tetraethyl orthosilicate was firstly added dropwise through a burette into a solution containing 100 mL 0.1 mol L⁻¹ tetrapropylammonium hydroxide and 5.4020 g aluminum nitrate. Then, the solution was further stirred and put in a 500 mL teflon lined autoclave. Next, the autoclave was sealed and heated up to 170° C. and held for 72 hours for hydrothermal synthesis. After the hydrothermal treatment, the product was collected and rinsed three times by deionized water through centrifugation. Finally, the catalyst structure was obtained through calcination at 600° C. in static air for 3 hours to get UZSM-5. KUZSM-5 catalyst was prepared from UZSM-5 through an ion exchange process. In a typical ion exchange cycle, 1 g UZSM-5 was put in 50 mL 0.01 mol L⁻¹ KOH aqueous solution and set for 5 minutes at room temperature, followed by a centrifugation process to separate the catalyst and the solution. The ion exchange cycle was repeated three times, and the final product was fully rinsed by deionized water and calcined at 600° C. in static air for 3 hours to get KUZSM-5.

The following metal salts were dissolved in deionized water to form a metal precursor solution: (NH₄)₆Mo₇O₂₄.4H₂O and Co(NO₃)₂.6H₂O. The KUZSM-5 support was impregnated with the metal precursor solution to achieve a suitable metal weight loading of 20 wt % Mo and 10 wt % Co. The obtained wet powder was first dried in an oven at 90° C. overnight, followed by calcination at 550° C. in static air for 2 hours to get the 20Mo10Co/KUZSM-5 catalyst.

The catalytic cyclohexene conversion was carried out in a batch reactor at 400° C. under a gas atmosphere of 32 bar CH₄ and 3 bar N₂ for 1 hour. First, 0.2000 g 20Mo10Co/KUZSM-5 catalyst and 2.00 g cyclohexene were put in a 100 mL batch reactor. Then, the reactor was sealed by flange with a reactor cap. Next, CH₄ was used to purge the reactor three times through a connected tubing system so that the other components in the air could be fully removed from the reactor. Subsequently, the reactor was set into a heating mantle and heated up to 400° C. after passing a leak test. The heating up stage took about 20 minutes and the reaction lasted another 1 hour after the temperature reached the set point. When the scheduled reaction time was met, the reactor was cooled down by blowing air flow at room temperature. The overall analysis, PONA selectivity and specific olefin selectivity are listed in Table 1, Table 2 and Table 3 respectively.

### Example 13

An NH₄-ZSM-5 (SiO₂:Al₂O₃=80:1) catalyst structure in powder form was obtained from Zeolyst USA. The catalyst was calcined in static air at 600° C. for 3 hours to get the HZSM-5(80:1) catalyst. KZSM-5 catalyst was prepared from ZSM-5 through an ion exchange process. In a typical ion exchange cycle, 1 g ZSM-5 was put in 50 mL 0.01 mol L⁻¹ KOH aqueous solution and set for 5 minutes at room temperature, followed by a centrifugation process to separate the catalyst and the solution. The ion exchange cycle was repeated three times, and the final product was fully rinsed by deionized water and calcined at 600° C. in static air for 3 hours to get KZSM-5.

The following metal salts were dissolved in deionized water to form a metal precursor solution: (NH₄)₆Mo₇O₂₄.4H₂O and Co(NO₃)₂.6H₂O. The KZSM-5 support was impregnated with the metal precursor solution to achieve a suitable metal weight loading of 20 wt % Mo and 10 wt % Co. The obtained wet powder was first dried in an oven at 90° C. overnight, followed by calcination at 550° C. in static air for 2 hours to get the 20Mo10Co/KZSM-5 catalyst.

The catalytic cyclohexene conversion was carried out in a batch reactor at 350° C. under a gas atmosphere of 32 bar CH₄ and 3 bar N₂ for 1 hour. First, 0.2000 g 20Mo10Co/KZSM-5 catalyst and 2.00 g cyclohexene were put in a 100 mL batch reactor. Then, the reactor was sealed by flange with a reactor cap. Next, CH₄ was used to purge the reactor three times through a connected tubing system so that the other components in the air could be fully removed from the reactor. Subsequently, the reactor was set into a heating mantle and heated up to 350° C. after passing a leak test. The heating up stage took about 20 minutes and the reaction lasted another 1 hour after the temperature reached the set point. When the scheduled reaction time was met, the reactor was cooled down by blowing air flow at room temperature. The overall analysis, PONA selectivity and specific olefin selectivity are listed in Table 1, Table 2 and Table 3 respectively.

### Example 14

A home-made uniform zeolite catalyst UZSM-5 with SiO₂ to Al₂O₃ molar ratio of 80:1 was prepared in the following procedures. 120 g tetraethyl orthosilicate was firstly added dropwise through a burette into a solution containing 100 mL 0.1 mol L⁻¹ tetrapropylammonium hydroxide and 5.4020 g aluminum nitrate. Then, the solution was further stirred and put in a 500 mL teflon lined autoclave. Next, the autoclave was sealed and heated up to 170° C. and held for 72 hours for hydrothermal synthesis. After the hydrothermal treatment, the product was collected and rinsed three times by deionized water through centrifugation. Finally, the catalyst structure was obtained through calcination at 600° C. in static air for 3 hours to get UZSM-5. NaUZSM-5 catalyst was prepared from UZSM-5 through an ion exchange process. In a typical ion exchange cycle, 1 g UZSM-5 was put in 50 mL 0.01 mol L⁻¹ NaOH aqueous solution and set for 5 minutes at room temperature, followed by a centrifugation process to separate the catalyst and the solution. The ion exchange cycle was repeated three times, and the final product was fully rinsed by deionized water and calcined at 600° C. in static air for 3 hours to get NaUZSM-5.

The following metal salts were dissolved in deionized water to form a metal precursor solution: (NH₄)₆Mo₇O₂₄.4H₂O. The NaUZSM-5 support was impregnated with the metal precursor solution to achieve a suitable metal weight loading of 10 wt % Mo. The obtained wet powder was first dried in an oven at 90° C. overnight, followed by calcination at 550° C. in static air for 2 hours to get the 10Mo/NaUZSM-5 catalyst.

The catalytic cyclohexene conversion was carried out in a batch reactor at 400° C. under a gas atmosphere of 32 bar CH₄ and 3 bar N₂ for 1 hour. First, 0.2000 g 10Mo/NaUZSM-5 catalyst and 2.00 g cyclohexene were put in a 100 mL batch reactor. Then, the reactor was sealed by flange with a reactor cap. Next, CH₄ was used to purge the reactor three times through a connected tubing system so that the other components in the air could be fully removed from the reactor. Subsequently, the reactor was set into a heating mantle and heated up to 400° C. after passing a leak test. The heating up stage took about 20 minutes and the reaction lasted another 1 hour after the temperature reached the set point. When the scheduled reaction time was met, the reactor was cooled down by blowing air flow at room temperature. The overall analysis, PONA selectivity and specific olefin selectivity are listed in Table 1, Table 2 and Table 3 respectively.

### Example 15

A home-made uniform zeolite catalyst UZSM-5 with SiO₂ to Al₂O₃ molar ratio of 80:1 was prepared in the following procedures. 120 g tetraethyl orthosilicate was firstly added dropwise through a burette into a solution containing 100 mL 0.1 mol L⁻¹ tetrapropylammonium hydroxide and 5.4020 g aluminum nitrate. Then, the solution was further stirred and put in a 500 mL teflon lined autoclave. Next, the autoclave was sealed and heated up to 170° C. and held for 72 hours for hydrothermal synthesis. After the hydrothermal treatment, the product was collected and rinsed three times by deionized water through centrifugation. Finally, the catalyst structure was obtained through calcination at 600° C. in static air for 3 hours to get UZSM-5. NaUZSM-5 catalyst was prepared from UZSM-5 through an ion exchange process. In a typical ion exchange cycle, 1 g UZSM-5 was put in 50 mL 0.01 mol L⁻¹ NaOH aqueous solution and set for 5 minutes at room temperature, followed by a centrifugation process to separate the catalyst and the solution. The ion exchange cycle was repeated three times, and the final product was fully rinsed by deionized water and calcined at 600° C. in static air for 3 hours to get NaUZSM-5.

The following metal salts were dissolved in deionized water to form a metal precursor solution: CoCl₂.6H₂O. The NaUZSM-5 support was impregnated with the metal precursor solution to achieve a suitable metal weight loading of 10 wt % Co. The obtained wet powder was first dried in an oven at 90° C. overnight, followed by calcination at 550° C. in static air for 2 hours to get the 10Co/NaUZSM-5 catalyst.

The catalytic cyclohexene conversion was carried out in a batch reactor at 400° C. under a gas atmosphere of 32 bar CH₄ and 3 bar N₂ for 1 hour. First, 0.2000 g 10Co/NaUZSM-5 catalyst and 2.00 g cyclohexene were put in a 100 mL batch reactor. Then, the reactor was sealed by flange with a reactor cap. Next, CH₄ was used to purge the reactor three times through a connected tubing system so that the other components in the air could be fully removed from the reactor. Subsequently, the reactor was set into a heating mantle and heated up to 400° C. after passing a leak test. The heating up stage took about 20 minutes and the reaction lasted another 1 hour after the temperature reached the set point. When the scheduled reaction time was met, the reactor was cooled down by blowing air flow at room temperature. The overall analysis, PONA selectivity and specific olefin selectivity are listed in Table 1, Table 2 and Table 3 respectively.

### Example 16

A home-made uniform zeolite catalyst UZSM-5 with SiO₂ to Al₂O₃ molar ratio of 80:1 was prepared in the following procedures. 120 g tetraethyl orthosilicate was firstly added dropwise through a burette into a solution containing 100 mL 0.1 mol L⁻¹ tetrapropylammonium hydroxide and 5.4020 g aluminum nitrate. Then, the solution was further stirred and put in a 500 mL teflon lined autoclave. Next, the autoclave was sealed and heated up to 170° C. and held for 72 hours for hydrothermal synthesis. After the hydrothermal treatment, the product was collected and rinsed three times by deionized water through centrifugation. Finally, the catalyst structure was obtained through calcination at 600° C. in static air for 3 hours to get UZSM-5. NaUZSM-5 catalyst was prepared from UZSM-5 through an ion exchange process. In a typical ion exchange cycle, 1 g UZSM-5 was put in 50 mL 0.01 mol L⁻¹ NaOH aqueous solution and set for 5 minutes at room temperature, followed by a centrifugation process to separate the catalyst and the solution. The ion exchange cycle was repeated three times, and the final product was fully rinsed by deionized water and calcined at 600° C. in static air for 3 hours to get NaUZSM-5.

The catalytic cyclohexene conversion was carried out in a batch reactor at 400° C. under a gas atmosphere of 32 bar CH₄ and 3 bar N₂ for 1 hour. First, 0.2000 g NaUZSM-5 catalyst and 2.00 g cyclohexene were put in a 100 mL batch reactor. Then, the reactor was sealed by flange with a reactor cap. Next, CH₄ was used to purge the reactor three times through a connected tubing system so that the other components in the air could be fully removed from the reactor. Subsequently, the reactor was set into a heating mantle and heated up to 400° C. after passing a leak test. The heating up stage took about 20 minutes and the reaction lasted another 1 hour after the temperature reached the set point. When the scheduled reaction time was met, the reactor was cooled down by blowing air flow at room temperature. The overall analysis, PONA selectivity and specific olefin selectivity are listed in Table 1, Table 2 and Table 3 respectively.

### Example 17

A home-made uniform zeolite catalyst UZSM-5 with SiO₂ to Al₂O₃ molar ratio of 80:1 was prepared in the following procedures. 120 g tetraethyl orthosilicate was firstly added dropwise through a burette into a solution containing 100 mL 0.1 mol L⁻¹ tetrapropylammonium hydroxide and 5.4020 g aluminum nitrate. Then, the solution was further stirred and put in a 500 mL teflon lined autoclave. Next, the autoclave was sealed and heated up to 170° C. and held for 72 hours for hydrothermal synthesis. After the hydrothermal treatment, the product was collected and rinsed three times by deionized water through centrifugation. Finally, the catalyst structure was obtained through calcination at 600° C. in static air for 3 hours to get UZSM-5.

The catalytic cyclohexene conversion was carried out in a batch reactor at 400° C. under a gas atmosphere of 32 bar CH₄ and 3 bar N₂ for 1 hour. First, 0.2000 g UZSM-5 catalyst and 2.00 g cyclohexene were put in a 100 mL batch reactor. Then, the reactor was sealed by flange with a reactor cap. Next, CH₄ was used to purge the reactor three times through a connected tubing system so that the other components in the air could be fully removed from the reactor. Subsequently, the reactor was set into a heating mantle and heated up to 400° C. after passing a leak test. The heating up stage took about 20 minutes and the reaction lasted another 1 hour after the temperature reached the set point. When the scheduled reaction time was met, the reactor was cooled down by blowing air flow at room temperature. The overall analysis, PONA selectivity and specific olefin selectivity are listed in Table 1, Table 2 and Table 3 respectively.

### Example 18

An NH₄-ZSM-5 (SiO₂:Al₂O₃=80:1) catalyst structure in powder form was obtained from Zeolyst USA. The catalyst was calcined in static air at 600° C. for 3 hours to get the HZSM-5(80:1) catalyst. NaZSM-5 catalyst was prepared from ZSM-5 through an ion exchange process. In a typical ion exchange cycle, 1 g ZSM-5 was put in 50 mL 0.01 mol L⁻¹ NaOH aqueous solution and set for 5 minutes at room temperature, followed by a centrifugation process to separate the catalyst and the solution. The ion exchange cycle was repeated three times, and the final product was fully rinsed by deionized water and calcined at 600° C. in static air for 3 hours to get NaZSM-5.

The catalytic cyclohexene conversion was carried out in a batch reactor at 400° C. under a gas atmosphere of 1 atm N₂ for 1 hour. First, 0.2000 g NaZSM-5 catalyst and 2.00 g cyclohexene were put in a 100 mL batch reactor. Then, the reactor was sealed by flange with a reactor cap. Next, CH₄ was used to purge the reactor three times through a connected tubing system so that the other components in the air could be fully removed from the reactor. Subsequently, the reactor was set into a heating mantle and heated up to 400° C. after passing a leak test. The heating up stage took about 20 minutes and the reaction lasted another 1 hour after the temperature reached the set point. When the scheduled reaction time was met, the reactor was cooled down by blowing air flow at room temperature. The overall analysis, PONA selectivity and specific olefin selectivity are listed in Table 1, Table 2 and Table 3 respectively.

TABLE 1
Overall analysis in example 1-18

| | | | | Gas | Liquid | Coke | Overall mass |
|---|---|---|---|---|---|---|---|
| | | | Cyclohexene | yield | yield | yield | balance |
| Example | Catalyst | Gas environment | conv. (%) | (wt %) | (wt %) | (wt %) | (%) |
| 1 | ZSM-5(23:1) | 30 bar CH₄ | 99.4 | 12.1 | 86.1 | 1.4 | 99.6 |
| 2 | ZSM-5(80:1) | 30 bar CH₄ | 100 | 12.3 | 88.5 | 1.3 | 102.1 |
| 3 | ZSM-5(280:1) | 30 bar CH₄ | 99.7 | 10.8 | 88.7 | 0.5 | 100.0 |
| 4 | 1Ag1Ga/ZSM-5 | 30 bar CH₄ | 98.7 | 10.3 | 88.2 | 2.6 | 101.1 |
| 5 | 20Mo/ZSM-5 | 30 bar CH₄ | 99.1 | 4.8 | 92.5 | 1.1 | 98.4 |
| 6 | 20Mo7.4Co/ZSM-5 | 30 bar CH₄ | 61.5 | 0.7 | 98.5 | 0.8 | 100.0 |
| 7 | 20Mo10Ce/ZSM-5 | 30 bar CH₄ | 96.1 | 5.9 | 94.6 | 0.9 | 101.4 |
| 8 | 20Mo7.4Co10Ce | 30 bar CH₄ | 32.6 | 0.5 | 95.7 | 0.6 | 96.8 |
| | 0.1Ag0.1Ga/ZSM-5 | | | | | | |
| 9 | 10Co10Mo/UZSM-5 | 32 bar CH₄, 3 bar N₂ | 63.8 | 2.5 | 96.3 | 1.4 | 100.2 |
| 10 | 10Col0Mo/ | 32 bar CH₄, 3 bar N₂ | 52.2 | 0.2 | 98.7 | 1.3 | 100.2 |
| | NaUZSM-5 | | | | | | |
| 11 | 10Mo10Co/ | 32 bar CH₄, 3 bar N₂ | 79.6 | 2.0 | 96.3 | 0.9 | 99.2 |
| | NaZSM-5 | | | | | | |
| 12 | 10Mo10Co/ | 32 bar CH₄, 3 bar N₂ | 44.4 | 1.5 | 95.2 | 1.2 | 97.9 |
| | KUZSM-5 | | | | | | |
| 13 | 10Mo10Co/KZSM-5 | 32 bar CH₄, 3 bar N₂ | 26.2 | 5.5 | 91.9 | 2.4 | 99.8 |
| 14 | 10Mo/NaUZSM-5 | 32 bar CH₄, 3 bar N₂ | 96.2 | 8.0 | 89.5 | 0.8 | 98.3 |
| 15 | 10Co/NaUZSM-5 | 32 bar CH₄, 3 bar N₂ | 46.8 | 1.5 | 96.8 | 0.4 | 98.8 |
| 16 | NaUZSM-5 | 32 bar CH₄, 3 bar N₂ | 44.0 | 3.3 | 94.8 | 0.4 | 98.5 |
| 17 | UZSM-5 | 32 bar CH₄, 3 bar N₂ | 93.0 | 6.0 | 91.2 | 0.9 | 98.1 |
| 18 | NaZSM-5 | 1 atm N₂ | 100 | 10.0 | 88.8 | 1.2 | 100 |

TABLE 2
PONA selectivity in example 1-22

| | | Gas | Paraffin | Olefin | Naphthene | Aromatic |
|---|---|---|---|---|---|---|
| Example | Catalyst | environment | (wt %) | (wt %) | (wt %) | (wt %) |
| 1 | ZSM-5(23:1) | 30 bar CH₄ | 10.58 | 1.41 | 1.80 | 86.21 |
| 2 | ZSM-5(80:1) | 30 bar CH₄ | 1.11 | 0.00 | 0.63 | 98.26 |
| 3 | ZSM-5(280:1) | 30 bar CH₄ | 4.62 | 0.00 | 0.68 | 94.71 |
| 4 | 1Ag1Ga/ZSM-5 | 30 bar CH₄ | 1.70 | 0.00 | 0.89 | 97.41 |
| 5 | 20Mo/ZSM-5 | 30 bar CH₄ | 9.57 | 6.59 | 2.89 | 80.87 |
| 6 | 20Mo7.4Co/ZSM-5 | 30 bar CH₄ | 8.04 | 57.85 | 4.01 | 30.25 |
| 7 | 20Mo10Ce/ZSM-5 | 30 bar CH₄ | 12.52 | 16.75 | 5.06 | 65.68 |
| 8 | 20Mo7.4Co10Ce | 30 bar CH₄ | 8.37 | 83.02 | 8.61 | 0.00 |
| | 0.1Ag0.1Ga/ZSM-5 | | | | | |
| 9 | 10Co10Mo/UZSM-5 | 32 bar CH₄, 3 bar N₂ | 2.65 | 56.65 | 3.66 | 37.05 |
| 10 | 10Co10Mo/ | 32 bar CH₄, | 2.18 | 95.33 | 2.48 | 0.00 |
| | NaUZSM-5 | 3 bar N₂ | | | | |
| 11 | 10Mo10Co/NaZSM-5 | 32 bar CH₄, | 0.79 | 78.73 | 2.45 | 18.02 |
| | | 3 bar N₂ | | | | |
| 12 | 10Mo10Co/ | 32 bar CH₄, | 2.25 | 86.76 | 5.36 | 5.63 |
| | KUZSM-5 | 3 bar N₂ | | | | |
| 13 | 10Mo10Co/KZSM-5 | 32 bar CH₄, | 1.72 | 87.39 | 10.54 | 0.36 |
| | | 3 bar N₂ | | | | |
| 14 | 10Mo/NaUZSM-5 | 32 bar CH₄, | 0.52 | 37.37 | 8.48 | 53.62 |
| | | 3 bar N₂ | | | | |
| 15 | 10Co/NaUZSM-5 | 32 bar CH₄, | 0.00 | 96.20 | 2.24 | 1.55 |
| | | 3 bar N₂ | | | | |
| 16 | NaUZSM-5 | 32 bar CH₄, | 0.00 | 94.70 | 3.52 | 1.78 |
| | | 3 bar N₂ | | | | |
| 17 | UZSM-5 | 32 bar CH₄, | 1.10 | 65.71 | 7.89 | 26.30 |
| | | 3 bar N₂ | | | | |
| 18 | NaZSM-5 | 1 atm N₂ | 2.00 | 1.70 | 1.01 | 96.29 |

TABLE 3
Olefin selectivity in example 1-22

| | | | | | | Other |
|---|---|---|---|---|---|---|
| | | Gas | 1-mcp | 3-mcp | 4-mcp | olefins |
| Example | Catalyst | environment | (wt %) | (wt %) | (wt %) | (wt %) |
| 1 | ZSM-5(23:1) | 30 bar CH₄ | 0.00 | 0.00 | 0.00 | 1.41 |
| 2 | ZSM-5(80:1) | 30 bar CH₄ | 0.00 | 0.00 | 0.00 | 0.00 |
| 3 | ZSM-5(280:1) | 30 bar CH₄ | 0.00 | 0.00 | 0.00 | 0.00 |
| 4 | 1Ag1Ga/ZSM-5 | 30 bar CH₄ | 0.00 | 0.00 | 0.00 | 0.00 |
| 5 | 20Mo/ZSM-5 | 30 bar CH₄ | 1.03 | 0.44 | 0.09 | 5.03 |
| 6 | 20Mo7.4Co/ZSM-5 | 30 bar CH₄ | 30.40 | 18.15 | 1.16 | 8.15 |
| 7 | 20Mo10Ce/ZSM-5 | 30 bar CH₄ | 6.53 | 3.11 | 0.42 | 6.67 |
| | 20Mo7.4Co10Ce | | | | | |
| 8 | 20Mo7.4Co10Ce | 30 bar CH₄ | 44.77 | 20.59 | 2.38 | 15.28 |
| | 0.1Ag0.1Ga/ZSM-5 | | | | | |
| 9 | 10Co10Mo/UZSM-5 | 32 bar CH₄, | 44.99 | 7.78 | 3.88 | 0.00 |
| | | 3 bar N₂ | | | | |
| 10 | 10Co10Mo/ | 32 bar CH₄, | 68.47 | 15.95 | 7.79 | 3.20 |
| | NaUZSM-5 | 3 bar N₂ | | | | |
| 11 | 10Mo10Co/NaZSM-5 | 32 bar CH₄, | 63.06 | 10.01 | 5.08 | 0.59 |
| | | 3 bar N₂ | | | | |
| 12 | 10Mo10Co/ | 32 bar CH₄, | 63.21 | 13.24 | 6.19 | 4.12 |
| | KUZSM-5 | 3 bar N₂ | | | | |
| 13 | 10Mo10Co/KZSM-5 | 32 bar CH₄, | 63.83 | 8.07 | 3.56 | 11.92 |
| | | 3 bar N₂ | | | | |
| 14 | 10Mo/NaUZSM-5 | 32 bar CH₄, | 28.28 | 5.46 | 2.52 | 1.11 |
| | | 3 bar N₂ | | | | |
| 15 | 10Co/NaUZSM-5 | 32 bar CH₄, | 79.41 | 10.79 | 4.99 | 0.99 |
| | | 3 bar N₂ | | | | |
| 16 | NaUZSM-5 | 32 bar CH₄, | 69.74 | 14.88 | 7.07 | 3.01 |
| | | 3 bar N₂ | | | | |
| 17 | UZSM-5 | 32 bar CH₄, | 48.73 | 10.23 | 4.49 | 2.26 |
| | | 3 bar N₂ | | | | |
| 18 | NaZSM-5 | 1 atm N₂ | 0.00 | 0.00 | 0.00 | 0.70 |

It can be seen that over 90 wt % liquid yield, less than 0.5 wt % gas yield, less than 0.5 wt % coke yield and over 95 wt % methylcyclopentene can be achieved with the provided catalyst structure, which provides a promising process for cyclohexene valorization in terms of high value-added methylcyclopentene formation with high purity.

While the invention has been described in detail and with reference to specific embodiments thereof, it will be apparent to one skilled in the art that various changes and modifications can be made therein without departing from the spirit and scope thereof.

Thus, it is intended that the present invention covers the modifications and variations of this invention provided they come within the scope of the appended claims and their equivalents.

What is claimed:

1. A method for producing methylcyclopentene from cyclohexene via skeletal isomerization, the method comprising:
reacting cyclohexene within a reactor in the presence of a gas atmosphere and a catalyst structure, wherein the catalyst structure comprises a porous support structure and one or more metals loaded in the porous support structure, the porous support structure comprises an aluminosilicate material, and the one or more metals loaded in the porous support structure is selected from the group consisting of Na, K, Co, Mo, Ag, Ga and Ce.

2. The method of claim 1, wherein the porous support structure includes Co and/or Mo.

3. The method of claim 1, wherein the gas atmosphere comprises a pure gas or a mixture of two or more gases selected from the group consisting of nitrogen, helium, methane, and argon.

4. The method of claim 1, wherein the aluminosilicate material is selected from the group consisting of HZSM-5 type zeolite, L-type zeolite, HX type zeolite, and HY type zeolite.

5. The method of claim 1, wherein each metal loaded in the porous support structure is present in an amount from 0.1 wt % to 20 wt % by weight of the catalyst support structure.

6. The method of claim 5, wherein the one or more metal components is loaded in the porous support structure as one or more salts selected from the group consisting of hydroxides, chlorides, and nitrates.

7. The method of claim 1, wherein the catalyst structure is formed by:
dissolving one or more metal salts in water to form a metal precursor solution;
loading the metal precursor solution into the porous support structure;
drying the support structure loaded with metal precursors for a period of at least 2 hours at a temperature from 80° C. to 120° C.; and
calcining the dried support structure loaded with metal precursor at a temperature ranging from 300° C. to 700° C.

8. The method of claim 7, wherein the gas atmosphere of calcination comprises one or the combination of more than one of the following gases: nitrogen, helium, argon and air.

9. The method of claim 1, wherein the porous support structure is in powder form or in pellet form.

10. The method of claim 1, wherein the reactor comprises a batch reactor system or a continuous tubular reactor (CTR).

11. The method of claim 1, wherein the conditions within the reactor comprise a reaction temperature within the range of 350° C. to 450° C., and a pressure within the range of 1 atm to 35 atm.

12. The method of claim 1, wherein the reactor comprises a batch reactor, and a mass ratio of cyclohexene to catalyst structure is within the range of about 100:1 to about 1:1.

13. The method of claim 1, wherein the reactor comprises a continuous tubular reactor, and a liquid hourly space velocity (LHSV) of the cyclohexene is within the range of 1 h⁻¹ to 100 h⁻¹.

14. The method of claim 1, wherein conversion of cyclohexene within the reactor to methylcyclopentene and/or other reaction products exceeds 50 wt %.

15. The method of claim 1, wherein selectivity of methylcyclopentene exceeds 90 wt % from cyclohexene conversion within the reactor.
